(19) United States
(12) Patent Application Publication
(10) Pub. No.: US 2019/0053696 A1
(43) Pub. Date: Feb. 21, 2019

(54) ENDOSCOPE APPARATUS

(71) Applicant: OLYMPUS CORPORATION, Tokyo (JP)

(72) Inventors: Makoto IGARASHI, Tokyo (JP); Yoichiro SAKANOUE, Tokyo (JP)

(73) Assignee: OLYMPUS CORPORATION, Tokyo (JP)

(21) Appl. No.: 16/166,734

(22) Filed: Oct. 22, 2018

Related U.S. Application Data
(63) Continuation of application No. PCT/JP2017/007441, filed on Feb. 27, 2017.

(30) Foreign Application Priority Data
Jul. 6, 2016 (JP) 2016-134364

(51) Int. Cl. A61B 1/06; A61B 1/07

(52) U.S. Cl. CPC A61B 1/0646; A61B 2090/3618; A61B 1/07

(57) ABSTRACT

An endoscope apparatus includes a light source apparatus including an LED, and an optical filter. The LED, generates, upon supply of a predetermined drive current, light having a peak wavelength at 600 nm as illuminating light to be radiated to a subject, and generates, upon supply of a drive current that is different from the predetermined drive current, light having the peak wavelength shifted to a wavelength that is different from 600 nm. The optical filter is provided on an optical path for the illuminating light, the optical path extending from the LED to an image pickup section configured to receive light from the subject and generate an image pickup signal, and the optical filter is configured to remove light having a wavelength located farther than 595 nm on a wavelength axis in a shifting direction of shifting to a short wavelength, from light on the optical path.

### CROSS REFERENCE TO RELATED APPLICATION

This application is a continuation application of PCT/JP2017/007441 filed on Feb. 27, 2017 and claims benefit of Japanese Application No. 2016-134364 filed in Japan on Jul. 6, 2016, the entire contents of which are incorporated herein by this reference.

### BACKGROUND OF INVENTION

### 1. Field of the Invention

The present invention relates to an endoscope apparatus and specifically relates to an endoscope apparatus including a light emitting section configured to generate illuminating light having a predetermined peak wavelength.

### 2. Description of the Related Art

Conventionally, endoscope apparatuses that radiate illuminating light and obtain an endoscopic image of the inside of a body cavity have been widely used. A surgeon can make various diagnoses or perform necessary treatments while viewing an endoscopic image of a living tissue displayed on a monitor, using an endoscope apparatus.

An endoscope apparatus that serves as a living body observation system has a plurality of observation modes such as a normal light observation mode in which a living tissue is illuminated with illuminating light that is white light to observe the living tissue and a special light observation mode in which the living tissue is illuminated with illuminating light that is special light to observe the living tissue.

Also, heat light sources such as xenon light sources have been used as light sources of endoscope apparatuses, but in recent years, as disclosed in Japanese Patent Application Laid-Open Publication No. 2016-49447, as light sources for illuminating light, endoscope apparatuses using semiconductor light emitting elements have been proposed. An amount of light emitted by each semiconductor light emitting element varies depending on a drive current.

### SUMMARY OF THE INVENTION

An endoscope apparatus according to an aspect of the present invention includes: a first light emitting section configured to generate, upon supply of a predetermined drive current, light having a peak wavelength at a first wavelength as illuminating light to be radiated to a subject, and to generate, upon supply of a drive current that is different from the predetermined drive current, light with the peak wavelength shifted to a second wavelength that is different from the first wavelength; and a removal section provided on an optical path for the illuminating light, the optical path extending from the first light emitting section to an image pickup section, the removal section being configured to remove light having a wavelength located farther than the second wavelength in a shifting direction of shifting from the first wavelength toward the second wavelength on a wavelength axis.

### BRIEF DESCRIPTION OF THE DRAWINGS

FIG. 1 is a configuration diagram illustrating a major part of an endoscope apparatus according to a first embodiment of the present invention;

FIG. 2 is a diagram indicating an intensity of each wavelength band of light emitted from an LED unit 32 and variations of coefficients of absorption by oxyhemoglobin and hemoglobin relative to wavelengths, according to the first embodiment of the present invention;

FIG. 3 is a diagram illustrating a configuration of a mirror unit 34 according to the first embodiment of the present invention;

FIG. 4 is a graph indicating a spectral reflection characteristic of a DM 34c and a spectral transmission characteristic of an optical filter 51, according to the first embodiment of the present invention;

FIG. 5 is a diagram for describing an overall processing flow in a special light observation mode, according to the first embodiment of the present invention;

FIG. 6 is a diagram for describing a flow of action, processing and operation when a distal end portion 2c of an endoscope 2 is brought close to an object, according to the first embodiment of the present invention;

FIG. 7 is a diagram indicating that a peak wavelength of narrow-band light emitted by an LED 32d shifts to the short wavelength side with a decrease in drive current for the LED 32d, according to the first embodiment of the present invention;

FIG. 8 is a diagram illustrating a configuration of a mirror unit 34A according to a second embodiment of the present invention;

FIG. 9 is a graph indicating a spectral reflection characteristic of a DM 34cA, according to the second embodiment of the present invention;

FIG. 10 is a diagram for describing a configuration of a DM 71 for an LED 32d, according to a modification of the second embodiment of the present invention; and

FIG. 11 is a diagram for describing the configuration of the DM 71 for the LED 32d, according to the modification of the second embodiment of the present invention.

### DETAILED DESCRIPTION OF THE PREFERRED EMBODIMENT(S)

Embodiments of the present invention will be described below with reference to the drawings.

### First Embodiment

### (Configuration)

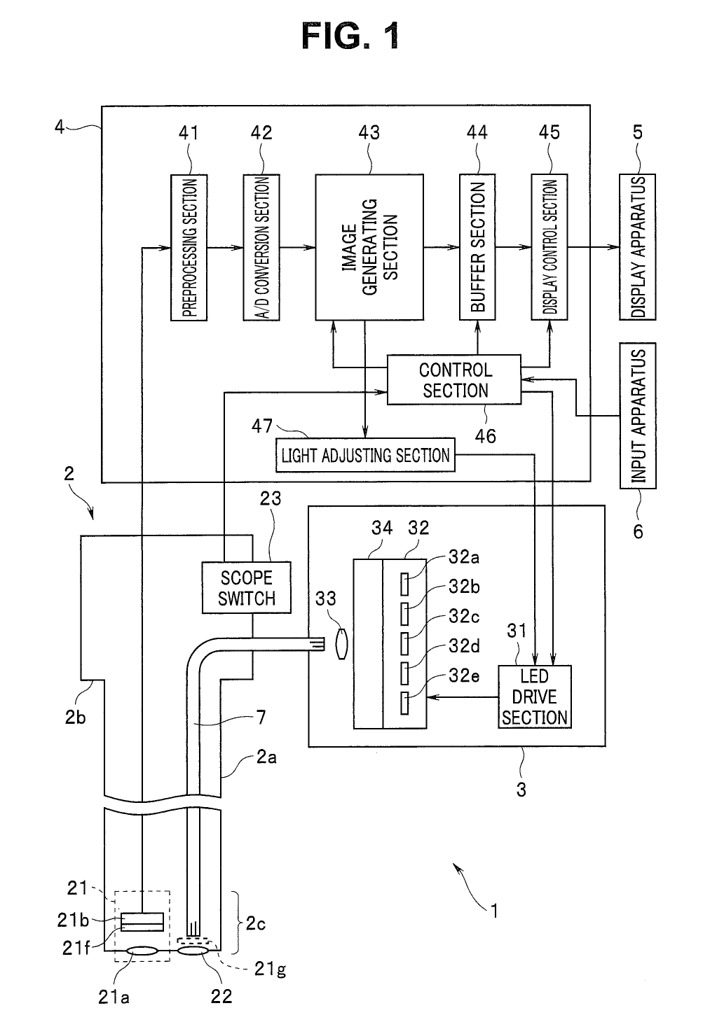

FIG. 1 is a configuration diagram illustrating a major part of an endoscope apparatus according to the present embodiment.

As illustrated in FIG. 1, an endoscope apparatus 1, which is a living body observation system, includes an endoscope 2, a light source apparatus 3, a processor 4, a display apparatus 5 and an input apparatus 6.

The endoscope 2 can be inserted into the inside of a subject and is configured to pick up an image of an object such as a living tissue inside the subject and output an image pickup signal. The light source apparatus 3 is configured to supply illuminating light to be used for observation of the object, via a light guide 7 inserted and arranged inside the endoscope 2. The processor 4 is configured to generate and output, e.g., a video signal according to the image pickup signal outputted from the endoscope 2. The display apparatus 5 displays, e.g., an observation image according to the video signal outputted from the processor 4. The input apparatus 6 includes, e.g., switches and/or buttons each capable of providing, e.g., an instruction according to an input operation performed by a user such as a surgeon to the processor 4.

The endoscope 2 includes an insertion portion 2a having an elongated shape that can be inserted into a subject, and an operation portion 2b provided on the proximal end side of the insertion portion 2a. Also, the endoscope 2 is configured to be detachably connected to the processor 4 via a universal cable (not illustrated) in which a plurality of signal wires to be used for transmission of various signals such as an image pickup signal are incorporated. Also, the endoscope 2 is configured to be detachably connected to the light source apparatus 3 via a light guide cable (not illustrated) in which at least a part of the light guide 7 is incorporated.

In the distal end portion 2c of the insertion portion 2a, an image pickup section 21 for picking up an image of an object such as a living tissue inside a subject, an output end portion of the light guide 7, and an illumination optical system 22 configured to radiate illuminating light transmitted by the light guide 7 to the object are provided.

The image pickup section 21 is configured to receive light from the object illuminated by the illuminating light outputted through the illumination optical system 22 and generate and output an image pickup signal. More specifically, the image pickup section 21 includes an objective optical system 21a configured to form an image of return light from the object, and an image pickup device 21b on which a color filter 21f of primary colors is disposed. The color filter 21f is arranged on a front face of a plurality of pixels for receiving the return light from the object and picking up an image of the return light, the plurality of pixels being arranged in a matrix at a position of an image formed via the objective optical system 21a.

The image pickup device 21b includes, for example, an image sensor such as a CCD or a CMOS, and is configured to generate an image pickup signal by picking up an image of return light passed through the color filter 21f and output the generated image pickup signal.

The color filter 21f is formed by arranging R (red), G (green) and B (blue) microscopic color filters at respective positions corresponding to respective pixels of the image pickup device 21b, in a mosaic-like fashion in a Bayer arrangement.

The operation portion 2b has a shape that enables a user to grasp and operate the operation portion 2b. Also, a scope switch 23 including one or more switches that each enable provision of an instruction according to an input operation performed by the user to the processor 4 is provided at the operation portion 2b.

The light source apparatus 3 includes an LED drive section 31, an LED unit 32, a condenser lens 33 and a mirror unit 34.

The LED drive section 31 includes, for example, a drive circuit. Also, the LED drive section 31 is configured to generate an LED drive signal for driving each LED in the LED unit 32 according to an illumination control signal and a light adjustment signal outputted from the processor 4 and output the LED drive signal.

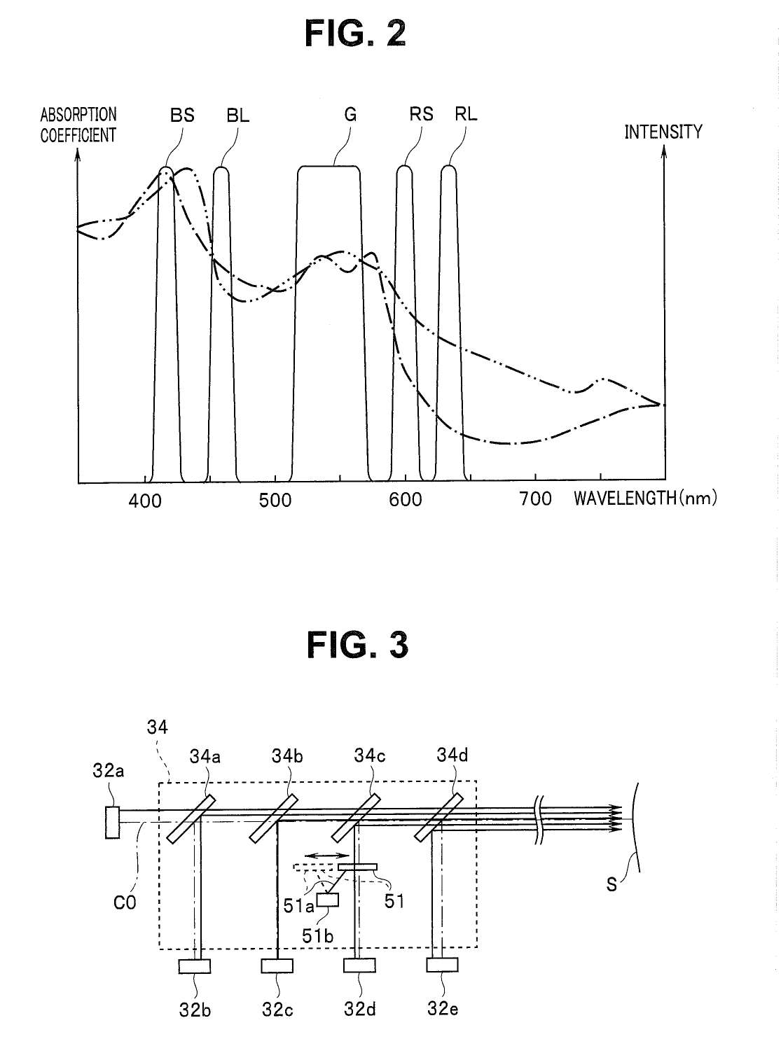

The LED unit 32 includes LEDs 32a to 32e, which are light sources configured to emit light having five mutually-different wavelength bands, for example, such as illustrated in FIG. 2. Also, the mirror unit 34 includes optical elements (see FIG. 3) such as dichroic mirrors for polarizing light emitted from the LEDs 32a to 32e to make the light enter the condenser lens 33.

FIG. 2 is a diagram indicating an intensity of each wavelength band of light emitted from the LED unit 32 and variations of coefficients of absorption by oxyhemoglobin and hemoglobin relative to wavelengths, according to the present embodiment.

The LEDs 32a to 32e are respective semiconductor light emitting elements configured to be individually turned on or off at respective timings according to LED drive signals outputted from the LED drive section 31. Also, the LEDs 32a to 32e are each configured to emit light having an emission intensity according to an LED drive signal outputted from the LED drive section 31.

For example, as illustrated in FIG. 2, the LED 32a has a center wavelength set to 415 nm and is configured to emit BS light, which is narrow-band light having a wavelength band set so as to belong to a blue range. In other words, BS light has a characteristic of being scattered and/or reflected by blood capillaries existing in the superficial layer of a living tissue and providing a high coefficient of absorption by blood in comparison with later-described BL light.

For example, as illustrated in FIG. 2, the LED 32b has a center wavelength set to 460 nm and is configured to emit BL light, which is narrow-band light having a wavelength band set so as to belong to the blue range. In other words, BL light has a characteristic of being scattered and/or reflected by blood capillaries existing in the superficial layer of a living tissue and providing a low coefficient of absorption by blood in comparison with BS light.

For example, as illustrated in FIG. 2, the LED 32c has a center wavelength set to 540 nm and is configured to emit G light, which is narrow-band light having a wavelength band set so as to belong to a green range. In other words, G light has a characteristic of being scattered and/or reflected by blood vessels existing in a middle layer on the superficial layer side relative to a deep part of a living tissue. Here, G light is narrow-band light having a relatively broad wavelength band including a wavelength band in a range other than the green range.

For example, as illustrated in FIG. 2, the LED 32d has a center wavelength set to 600 nm and is configured to emit RS light, which is narrow-band light having a wavelength band set so as to belong to a red range. In other words, RS light has a characteristic of being scattered and/or reflected by thick blood vessels existing in a deep part of a living tissue and providing a high coefficient of absorption by blood in comparison with later-described RL light.

For example, as illustrated in FIG. 2, the LED 32e has a center wavelength set to 630 nm and is configured to emit RL light, which is narrow-band light having a wavelength band set so as to belong to the red range. In other words, RL light has a characteristic of being scattered and/or reflected by thick blood vessels existing in a deep part of a living tissue and providing a low coefficient of absorption by blood in comparison with RS light.

An amount of light emitted from each semiconductor light emitting element varies according to a drive current. In each of the LEDs 32a to 32e, as a current value of the drive current becomes larger, a peak wavelength shifts to the long wavelength side, and as the current value of the drive current becomes smaller, the peak wavelength shifts to the short wavelength side. In particular, in the LED 32d, the peak wavelength shifts to the short wavelength side upon supply of a drive current having a current value that is smaller than a predetermined current value.

In other words, upon supply of a predetermined drive current, the LED 32d generates narrow-band light having a peak wavelength at a wavelength of no less than 600 nm, and upon supply of a drive current that is lower than the predetermined drive current, the LED 32d generates narrow-band light having a peak wavelength at a wavelength of less than 600 nm.

Therefore, the LED 32d configures a light emitting section configured to generate, upon supply of the predetermined drive current, light having a peak wavelength at a wavelength of 600 nm as illuminating light for illuminating a subject, and to generate, upon supply of a drive current that is different from the predetermined drive current, light having a peak wavelength shifted to a wavelength that is different from the wavelength of 600 nm, for example, 595 nm.

Each of the LEDs 32a, 32b and 32c is a light emitting section configured to generate light having a peak wavelength at a wavelength that is shorter than the wavelengths of light generated by the LED 32d. A DM 34c is arranged on an optical path on which light generated by the LED 32d and light generated by the LEDs 32a, 32b and 32c travel, and multiplexes the light from the LED 32d and the light from the LED 32a, etc.

A degree of absorption of light by hemoglobin largely varies in the vicinity of a wavelength of 600 nm.

In FIG. 2, the alternate long and short dash line indicates an absorption spectrum of oxyhemoglobin, and the alternate long and two short dashes line indicates an absorption spectrum of reduced hemoglobin.

For example, in general, venous blood contains oxyhemoglobin (HbO₂) and reduced hemoglobin (Hb) (hereinafter both collectively referred to simply as “hemoglobin”) at a ratio of approximately 60:40 to 80:20. Light is absorbed by hemoglobin, but a coefficient of the absorption differs by respective wavelengths of the light. A characteristic of absorption of light by venous blood at respective wavelengths of from approximately 400 nm to approximately 800 nm is that in a range of 550 to 750 nm, the absorption coefficient exhibits a local maximum value at a point that is substantially a wavelength of 576 nm and a local minimum value at a point of a wavelength of 730 nm.

RS light is narrow-band light having a peak wavelength of 600 nm, the peak wavelength being a center wavelength, and is light within a wavelength band of from a wavelength at which the absorption coefficient exhibits a local maximum value (here, the absorption coefficient at the wavelength of 576 nm) to a wavelength at which the absorption coefficient exhibits a local minimum value (here, the absorption coefficient at the wavelength of 730 nm) in the characteristic of absorption by hemoglobin.

RL light is narrow-band light having a peak wavelength of 630 nm, the peak wavelength being a center wavelength, and is light within a wavelength band of from a local maximum value to a local minimum value, the local maximum value and the local minimum value being the same as the above local maximum value and the above local minimum value, in the characteristic of absorption by hemoglobin, but RL light is light having a band of wavelengths that are longer than the wavelengths of RS light, in which the absorption coefficient is low, and the characteristic of being scattered by a living tissue is suppressed. The phrase “the characteristic of being scattered is suppressed” means that a scattering coefficient becomes lower toward the long wavelength side.

FIG. 3 is a diagram illustrating a configuration of the mirror unit 34.

The mirror unit 34 includes four dichroic mirrors (hereinafter referred to as DMs) 34a, 34b, 34c, 34d and an optical filter 51.

The DM 34a has a spectral reflection characteristic of reflecting light in a wavelength band of no less than 460 nm and a spectral transmission characteristic of transmitting light in a wavelength band of less than 460 nm. The DM 34a is arranged at a position at which the DM 34a reflects light emitted from the LED 32b and emits the light to an object S along an optical path C0 on which light emitted from the LED 32a is outputted to the object S, on the optical path C0.

The DM 34b has a spectral reflection characteristic of reflecting light of a wavelength band of no less than 540 nm and a spectral transmission characteristic of transmitting light of a wavelength band of less than 540 nm. The DM 34b is arranged at a position at which the DM 34b reflects light emitted from the LED 32c and emits the light to the object S along the optical path C0 on which light emitted from the LED 32a is outputted to the object S, on the optical path C0.

The DM 34c has a spectral reflection characteristic of reflecting light of a wavelength band of no less than 585 nm and a spectral transmission characteristic of transmitting light of a wavelength band of less than 585 nm. The DM 34c is arranged at a position at which the DM 34c reflects light emitted from the LED 32d and emits the light to the object S along the optical path C0 on which light emitted from the LED 32a is outputted to the object S, on the optical path C0.

The DM 34d has a spectral reflection characteristic of reflecting light of a wavelength band of no less than 630 nm and a spectral transmission characteristic of transmitting light of a wavelength band of less than 630 nm. The DM 34d is arranged at a position at which the DM 34d reflects light emitted from the LED 32e and outputs the light to the object S along the optical path C0 on which light emitted from the LED 32a is outputted to the object S, on the optical path C0.

The optical filter 51 is disposed between the LED 32d and the DM 34c.

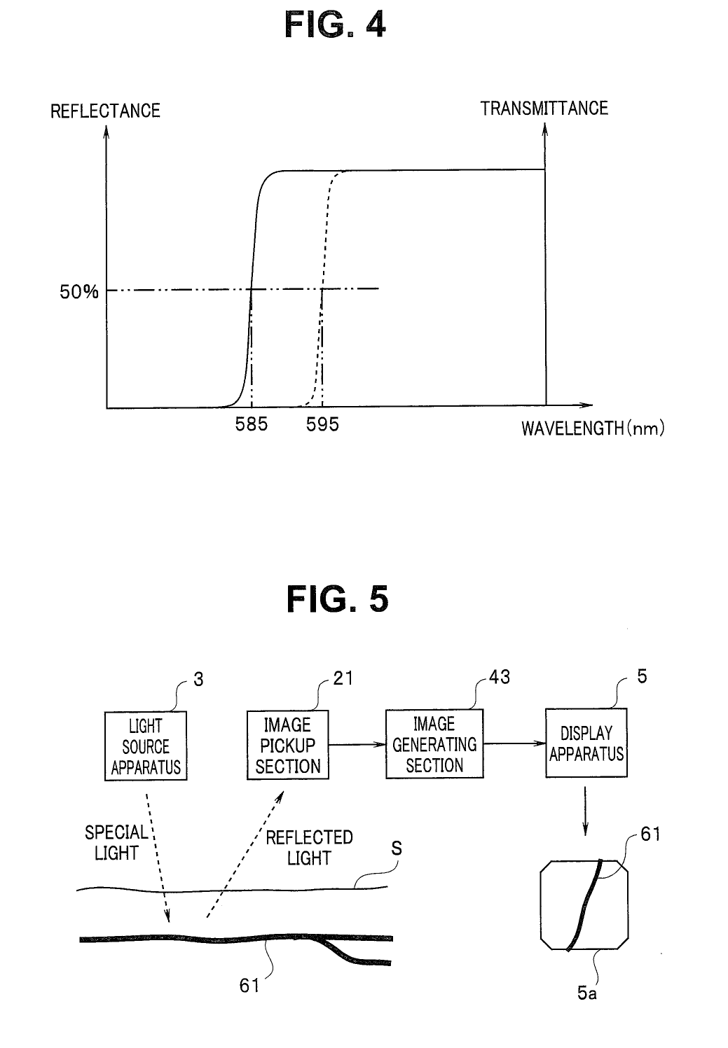

The optical filter 51 is a long pass filter configured to transmit light in a wavelength band of no less than 595 nm. FIG. 4 is a graph indicating a spectral reflection characteristic of the DM 34c and a spectral transmission characteristic of the optical filter 51.

The DM 34c reflects illuminating light emitted from the LED 32d so as to radiate the illuminating light toward the object; however, as indicated by the solid line in FIG. 4, the DM 34c reflects only light in a wavelength band of no less than 585 nm.

As indicated by the dotted line in FIG. 4, the optical filter 51 removes light in a wavelength band of no more than 595 nm.

As described above, the optical filter 51 is provided on an optical path for illuminating light extending from the LED 32d to the image pickup section 21, here, between the LED 32d and the DM 34c on an optical path from the LED 32d toward the object. The optical filter 51 configures a removal section configured to remove light having a wavelength located farther than 595 nm (that is, light having a wavelength band of no more than 595 nm) on the wavelength axis in a shifting direction of shifting from the wavelength of 600 nm to the wavelength of 595 nm (that is, the short wavelength side) from light on the optical path.

The optical filter 51 removes light having wavelengths of less than 600 nm, which is a peak wavelength, from illuminating light from the LED 32d, here, removes light having wavelengths that are less than 595 nm.

As illustrated in FIG. 3, the optical filter 51 is movable and is connected to an actuator 51b including, e.g., a motor, by an arm member 51a. The actuator 51b is controlled and driven by a control section 46 via the LED drive section 31.

In a special light observation mode, as indicated by the solid line in FIG. 3, the optical filter 51 is disposed between the LED 32d and the DM 34c. In a normal light observation mode, as indicated by the dotted line in FIG. 3, the optical filter 51 is moved to a position at which the optical filter 51 is not disposed between the LED 32d and the DM 34c. As indicated by the arrow in FIG. 3, the optical filter 51 is movable, and in the special light observation mode, is disposed between the LED 32d and the DM 34c as indicated by the solid line. In other words, the optical filter 51 is a removal section configured to remove light in a band of wavelengths that are not longer than a predetermined wavelength and is inserted/removed onto/from the optical path for illuminating light in response to selection of the observation mode via the input apparatus 6 or the scope switch 23.

The condenser lens 33 is configured to collect light outputted from the mirror unit 34 and make the light enter an input end portion of the light guide 7.

Referring back to FIG. 1, the processor 4 includes a preprocessing section 41, an A/D conversion section 42, an image generating section 43, a buffer section 44, a display control section 45, a control section 46 and a light adjusting section 47.

The preprocessing section 41 includes, for example, various processing circuits. Also, the preprocessing section 41 is configured to subject an image pickup signal outputted from the image pickup section 21 of the endoscope 2 to predetermined signal processing such as amplification and denoising and output the resulting image pickup signal to the A/D conversion section 42.

The A/D conversion section 42 includes, for example, an A/D conversion circuit. Also, the A/D conversion section 42 is configured to generate image data by subjecting the image pickup signal outputted from the preprocessing section 41 to processing such as A/D conversion and output the generated image data to the image generating section 43.

The image generating section 43 includes, for example, a color separation processing circuit and a color balance circuit. The image generating section 43 is configured to output the image data subjected to color balance processing and the like to the buffer section 44.

The buffer section 44 includes, e.g., a buffer circuit such as a buffer memory. Also, the buffer section 44 is configured to temporarily accumulate, under the control of the control section 46, the image data outputted from the image generating section 43 and output the accumulated image data to the display control section 45.

The display control section 45 includes, for example, a display control circuit. Also, the display control section 45 is configured to under the control of the control section 46, generate video signals by allocating the image data outputted from the buffer section 44 to an R channel, a G channel and a B channel of the display apparatus 5 and output the generated video signals to the display apparatus 5.

The control section 46 includes, for example, a control circuit including, e.g., a CPU, a ROM and a RAM. In the ROM, e.g., a program that controls overall operation of the endoscope apparatus 1 and programs that control operation according to respective observation modes are stored, and the CPU reads and executes various programs from the ROM in response to instructions from a user and executes the programs and outputs control signals to the respective sections.

The control section 46 is configured to generate an illumination control signal for illuminating an object, according to an observation mode and output the illumination control signal to the LED drive section 31.

The control section 46 is configured to control the display control section 45 to change an observation image displayed on the display apparatus 5, according to a desired observation mode selected from among a plurality of observation modes that can be selected via an observation mode selection switch (not illustrated) provided at the input apparatus 6 and/or the scope switch 23. Therefore, the input apparatus 6 or the scope switch 23 configures an observation mode selection section configured to select an observation mode for a subject.

The light adjusting section 47 includes, for example, a light adjusting circuit. Also, the light adjusting section 47 is configured to generate a light adjustment signal for adjusting an intensity of light emitted by each of the LEDs of the LED unit 32 based on the image data outputted from the image generating section 43 and output the generated light adjustment signals to the LED drive section 31.

### (Operation)

A surgeon can observe a subject in a desired observation mode by operating the observation mode selection switch provided at the input apparatus 6 and/or the scope switch 23.

Upon the observation mode being set to the normal light observation mode, the control section 46 controls the LED drive section 31 to cause the five LEDs 32a to 32e to emit light, and as indicated by the dotted line in FIG. 3, the optical filter 51 is moved to the position at which the optical filter 51 is not disposed between the LED 32d and the DM 34c.

Furthermore, the control section 46 controls the image generating section 43, the buffer section 44 and the display control section 45 to display an endoscopic image for normal light observation on the display apparatus 5, according to the normal light observation mode.

An endoscopic image in the normal light observation mode is generated from return light of five narrow-band light rays emitted from the five LEDs 32a to 32e.

Upon the observation mode being set to the special light observation mode, the control section 46 controls the LED drive section 31 to cause only three LEDs: one of the LED 32b and the LED 32c, the LED 32d and the LED 32e, from among the five LEDs 32a to 32e, to emit light and also causes the optical filter 51 to be moved between the LED 32d and the DM 34c as indicated by the solid line in FIG. 3.

Here, in the special light observation mode, three narrow-band images obtained from respective return light rays of the illuminating light of 460 nm (or 540 nm), the illuminating light of 600 nm and the illuminating light of 630 nm are allocated to three input channels, the blue channel, the green channel and the red channel, of the display apparatus 5, and the narrow-band images for deep blood vessel highlighted display or bleeding point display are displayed on a display screen 5a.

Here, the special light observation mode is a narrow-band light observation mode for deep blood vessel highlighted display or bleeding point display.

FIG. 5 is a diagram for describing an overall processing flow in the special light observation mode according to the present embodiment.

A surgeon inserts the insertion portion 2a of the endoscope into a body cavity and positions the distal end portion 2c of the insertion portion 2a at the vicinity of a lesion part and confirms the lesion part to be subjected to treatment in the normal observation mode. Then, the surgeon operates the observation mode selection switch to select the special light observation mode for the endoscope apparatus 1 in order to observe, for example, a relatively-thick submucosal deep blood vessel 61 having a diameter of 1 to 2 mm. Here, the deep blood vessel 61 is an observation target and is an object existing in a depth direction of the mucous membrane of a living body.

In the narrow-band observation mode, the control section 46 controls the LED drive section 31 of the light source apparatus 3 to emit predetermined three narrow-band light rays. In this case, as described above, the optical filter 51 is inserted between the LED 32d and the DM 34c as indicated by the solid line in FIG. 3. The control section 46 controls the respective circuits in the processor 4 to generate an endoscopic image for special light observation.

As illustrated in FIG. 5, in the special light observation mode, illuminating light having three narrow wavelength bands from the light source apparatus 3, which is a light emitting section, is outputted from the distal end portion 2c of the insertion portion 2a of the endoscope 2, penetrates the mucosal layer of an object S and irradiates the deep blood vessel 61 running in the submucosal layer and the proper muscular layer.

Reflected light of narrow-band light having a center wavelength of around 460 nm or 540 nm, narrow-band light having a center wavelength of around 600 nm and narrow-band light having a center wavelength of around 630 nm is received by the image pickup section 21. An image pickup signal outputted by the image pickup section 21 is supplied to the above-described image generating section 43.

An image signal generated as a result of processing in the image generating section 43 is outputted onto the display screen 5a of the display apparatus 5. On the display screen 5a, the deep blood vessel 61 is displayed in a highlighted manner or a bleeding point is displayed.

In the special light observation mode, upon the distal end portion 2c being brought close to the object, control of an amount of illuminating light is performed, and description will be given on the point that according to the endoscope apparatus 1 of the present embodiment, a bleeding point is displayed with no decrease in contrast.

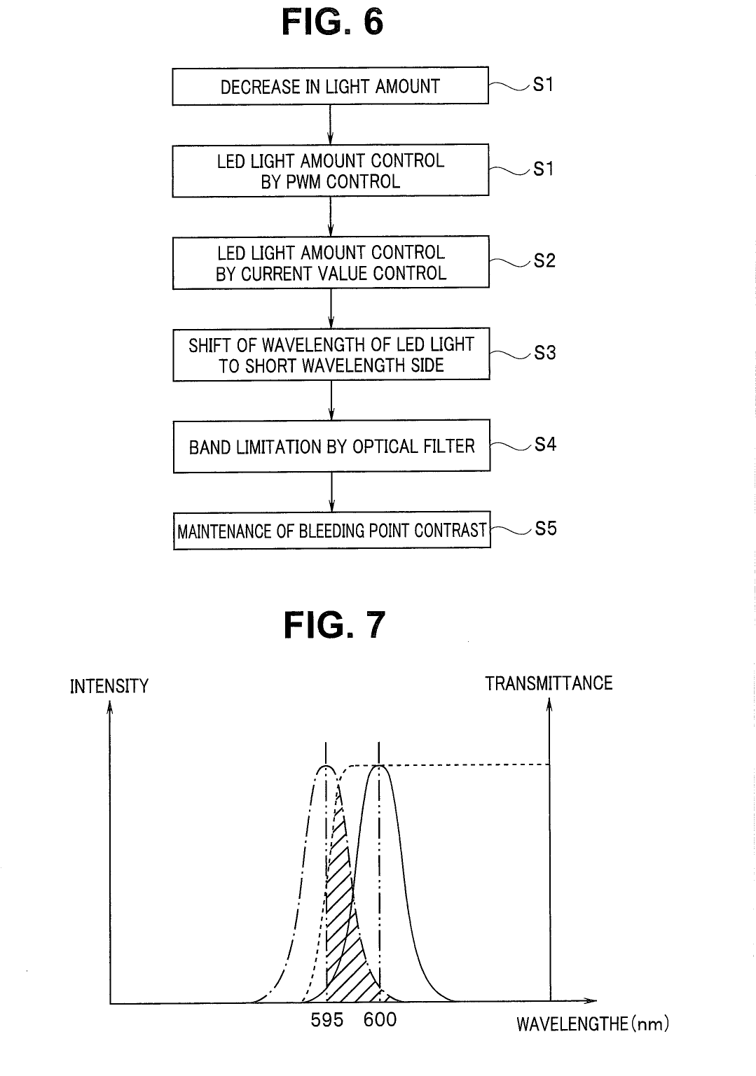

FIG. 6 is a diagram for describing a flow of action, processing and operation when the distal end portion 2c of the endoscope 2 is brought close to an object.

When the distal end portion 2c is brought close to an object, a decrease in amount of illuminating light by controlling the light adjusting section 47 becomes necessary (S0). In order to decrease the amount of illuminating light, three LEDs: one of the LED 32b and the LED 32c, the LED 32d and the LED 32e, are subjected to LED light amount control by PWM control in which light emission is turned on/off based on PWM (S1). In other words, in order to generate an endoscopic image of proper brightness, the light adjusting section 47 causes the LED drive section 31 to operate to drive the three LEDs based on PWM control.

Each LED emits narrow-band light having a predetermined peak wavelength upon supply of a predetermined drive current. In particular, the LED 32d emits narrow-band light having a peak wavelength of 600 nm upon supply of a predetermined current value P, for example, a maximum drive current value of drive current PI. During PWM control, supply of the predetermined current value P of the drive current PI is turned on/off according to a duty ratio.

When no light amount decrease can be made any longer only by adjustment of amount of illuminating light by PWM control as a result of the distal end portion 2c being brought closer to the object, LED light amount control by current value control is performed (S2).

In the case of PWM control, each of the three LEDs is subjected to on/off control at a calculated duty ratio, with the predetermined current value P (for example, the maximum current value) of the drive current PI flowing in the LED maintained as it is, and thus, neither decrease in current value of the drive current PI nor shifting of the peak wavelength of the LED 32d occurs.

Upon the current value control in S2 being performed, the current value of the drive current PI is decreased to a value p that is smaller than the predetermined current value P, and thus, shifting of the peak wavelength of the light from the LED 32d to the short wavelength side occurs (S3).

However, even if shifting of the peak wavelength of the light emitted by the LED 32d to the short wavelength side occurs, the optical filter 51 puts a band limitation so as to prevent penetration of light of no more than 595 nm.

FIG. 7 is a diagram indicating that a peak wavelength of narrow-band light emitted by the LED 32d shifts to the short wavelength side with a decrease in drive current for the LED 32d. As indicated by the solid line in FIG. 7, when the predetermined current value P, for example, a maximum current value Pmax of the drive current PI is supplied to the LED 32d, the LED 32d emits narrow-band light having a peak wavelength of 600 nm.

Upon a decrease in intensity, that is, current value of the drive current PI for the LED 32d, as indicated by the alternate long and short dash line in FIG. 7, the LED 32d emits narrow-band light with the peak wavelength shifted to the short wavelength side.

However, the optical filter 51 prevents penetration of light having a wavelength of less than 595 nm as indicated by the dotted line, and thus, light in the range shaded in FIG. 7 penetrates the optical filter 51.

As a result, only, light of no less than 595 nm, that is, narrow-band light of around 600 nm in the light from the LED 32d is reflected by the DM 34c and radiated to the object, and thus, contrasts of a deep blood vessel and a bleeding point displayed on the display screen 5a of the display apparatus 5 are maintained (S5).

Upon the peak wavelength being shifted to the short wavelength side, the light in the range shaded in FIG. 7 penetrates the optical filter 51. Therefore, upon the peak wavelength being shifted to the short wavelength side, the amount of narrow-band light of 600 nm slightly decreases, but according to a test conducted by the present application, a deep blood vessel and a bleeding point were displayed at a high contrast on the display screen 5a in such a manner that the deep blood vessel and the bleeding point have tints that are the same as tints of the deep blood vessel and the bleeding point in an image obtained when the predetermined current value P of the drive current PI was supplied.

Furthermore, according to a test conducted by the present applicant, even where the optical filter 51 has a spectral transmission characteristic of transmitting light of no less than 591 nm and not transmitting light of a wavelength of less than 591 nm, a deep blood vessel and a bleeding point were displayed at a high contrast on the display screen 5a in such a manner that the deep blood vessel and the bleeding point have tints that are the same as tints of the deep blood vessel and the bleeding point in an image obtained when the predetermined current value P of the drive current PI was supplied. Therefore, the optical filter 51, which serves as a removal section, may be configured to remove light having a wavelength of no more than 591 nm from illuminating light.

Also, since light of less than 595 nm is not radiated to the object, when a bleeding point is displayed, a part around the bleeding point has a reduced amount of blood and thus an amount of light of 600 nm absorbed by blood is small and no substantial difference occurs in how a mucosal membrane, through which blood can be seen, looks.

As above, in the above-described special light observation mode, a deep blood vessel can be displayed and a decrease in quality of the displayed image is suppressed, and when bleeding occurs during surgery, a point of the bleeding can be displayed. A surgeon identifies a position of the displayed bleeding point and performs hemostatic treatment of the bleeding point.

Upon a peak wavelength shifting according to a value of a drive signal, conventionally, a bleeding point fails to be displayed at a high contrast, but in the above-described special light observation mode, a contrast decrease is suppressed and thus color reproducibility of the bleeding point is good.

Also, when bleeding occurs during surgery, a deep blood vessel under a surrounding mucosal membrane that is away from a point of the bleeding is also displayed at a high contrast. Narrow-band light of 600 nm penetrates a thin blood layer spreading on the surrounding mucosal membrane and contains no light of a wavelength of no more than 595 nm. Therefore, the deep blood vessel under the mucosal membrane covered by the thin blood layer spreading on the surrounding mucosal membrane is also displayed at a high contrast.

Therefore, the above-described embodiment enables provision of an endoscope apparatus that even if a light source configured to emit illuminating light having a peak wavelength that shifts depending on a value of a drive signal is used, can reduce radiation of light of a wavelength unsuitable for desired observation.

Also, where specific narrow-band light is used for measurement of, e.g., oxygen saturation, an accurate measurement result can be obtained by provision of a removal section for limiting shifting of a peak wavelength of the narrow-band light such as described above.

Still furthermore, although the above embodiment indicates an example in which wavelength shifting to the short wavelength side occurs due to a decrease in value of a drive signal, a removal section may be provided in an example in which wavelength shifting to the long wavelength side occurs due to an increase in value of a drive signal.

### Second Embodiment

While in the first embodiment, image quality deterioration due to peak wavelength shifting is prevented using an optical filter configured not to transmit light in a wavelength band that is equal to or lower than a predetermined wavelength band, in the second embodiment, image quality deterioration due to peak wavelength shifting is prevented using a dichroic mirror (DM) configured not to reflect light in a wavelength band that is equal to or lower than a predetermined wavelength band.

Since an endoscope apparatus according to the second embodiment has a configuration that is substantially the same as the configuration of the endoscope apparatus 1 according to the first embodiment, and thus, in the endoscope apparatus according to the present embodiment, components that are the same as the components of the endoscope apparatus 1 according to the first embodiment are provided with reference numerals that are the same as the reference numerals of the components of the endoscope apparatus 1 and description of such components will be omitted.

The endoscope apparatus according to the present embodiment is substantially the same as the endoscope apparatus 1 according to the first embodiment illustrated in FIG. 1, but different from the endoscope apparatus 1 according to the first embodiment in configuration of a mirror unit.

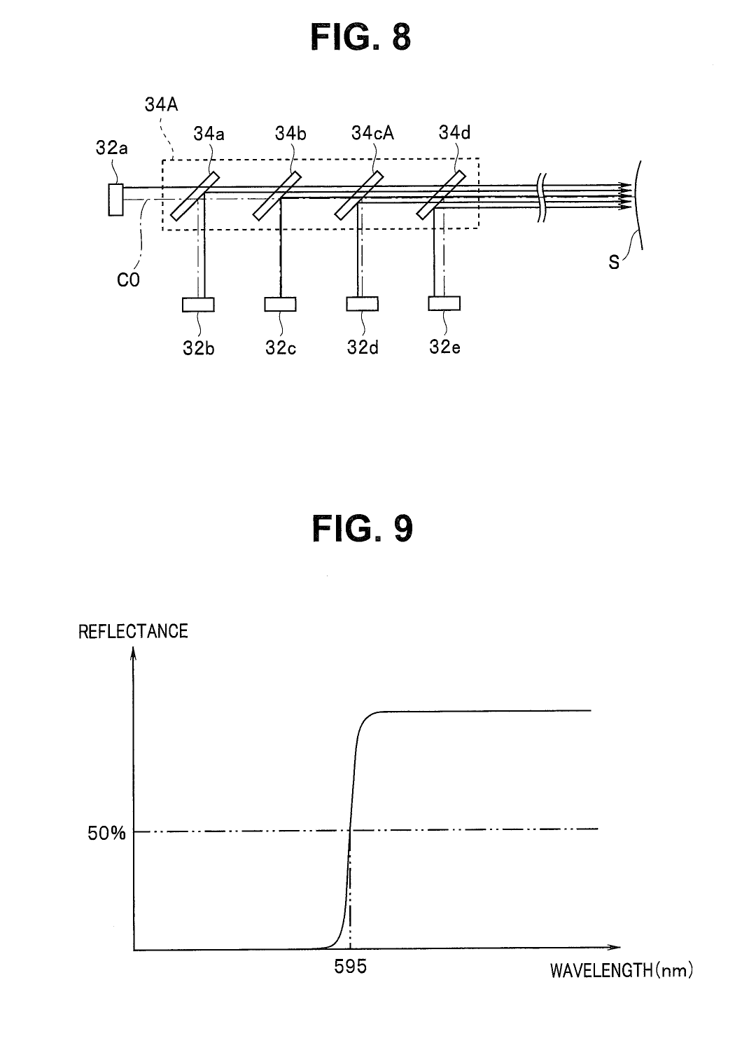

FIG. 8 is a diagram illustrating a configuration of a mirror unit 34A according to the present embodiment. FIG. 9 is a graph indicating spectral reflection characteristic of a DM 34cA.

A mirror unit 34A includes four DMs 34a, 34b, 34cA, 34d. The DM 34cA for an LED 32d has a spectral reflection characteristic of reflecting only light in a wavelength band of no less than 595 nm and a spectral transmission characteristic of transmitting light of a wavelength band of less than 595 nm. The DM 34cA is arranged at a position at which light emitted from the LED 32d is reflected and outputted to an object S along an optical path C0.

In other words, the DM 34cA configures a removal section arranged on an optical path on which light generated by the LED 32d and light generated by the LEDs 32a, 32b and 32c travel, the removal section being configured to not reflect light having a wavelength located farther than 595 nm on a wavelength axis in a direction of from 600 nm to 595 nm in the light from the LED 32d, but reflect only light in a wavelength band of no less than 595 nm and transmit the light from the LEDs 32a, 32b and 32c to multiplex the light from the LED 32d and the light from the LEDs 32a, 32b and 32c.

As a result, even if wavelengths of the light from the LED 32d are shifted to the short wavelength side, the DM 34cA puts a band limitation so as to prevent reflection of light having wavelengths of less than 595 nm, and thus, light in the range shaded in FIG. 7 is reflected and outputted to the object.

As a result, only light of no less than 595 nm, that is, narrow-band light of around 600 nm in the light from the LED 32d is reflected by the DM 34cA and radiated to the object, and thus, a contrast of, e.g., a bleeding point displayed on a display screen 5a of a display apparatus 5 is maintained.

The present embodiment also enables provision of effects that are similar to the effects of the first embodiment.

Next, a modification will be described.

### (Modification)

In the case of the second embodiment, in a normal light observation mode, light from the LED 32d is reflected by the DM 34cA, but upon shifting of a peak wavelength, an amount of light to be emitted to an object from the LED 32d decreases. Therefore, in the present modification, in order to prevent such decrease in amount of light from the LED 32d in the normal light observation mode, a DM selection is made according to an observation mode.

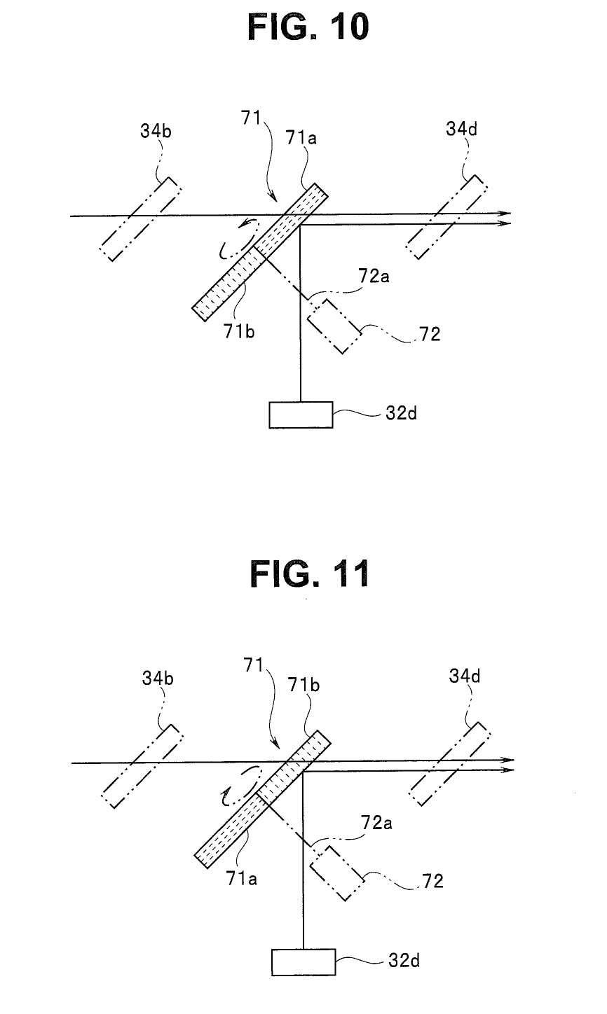

FIGS. 10 and 11 are diagrams for describing a configuration of a DM 71 for a LED 32d, according to the present modification. The DM 71 for the LED 32d includes two DMs having mutually-different reflection characteristics. Instead of the DM 34cA, the DM 71 is disposed between the DMs 34b and 34d.

The DM 71 includes two DMs 71a and 71b. As with the above-described DM 34c, the DM 71a has spectral reflection characteristic of reflecting light in a wavelength band of no less than 585 nm and transmitting light in a wavelength band of less than 585 nm. As with the above-described DM 34cA, the DM 71b has a spectral reflection characteristic of reflecting light of a wavelength band of no less than 595 nm and transmitting light of a wavelength band of less than 595 nm.

FIG. 10 illustrates a state in which the DM 71a of the DM 71 is arranged on an optical path C0 in the normal light observation mode, and FIG. 11 illustrates a state in which the DM 71b of the DM 71 is arranged on the optical path C0 in a special light observation mode.

The DM 71 has a disc shape, and the DM 71a and the DM 71b each have a semi-disc shape and are fixed to a shaft 72a of a motor 72. In response to driving of the motor 72, one of the DMs 71a and 71b can be arranged on the optical path C0.

Driving of the motor 72 is controlled by a control section 46, and the disc-shaped DM 71 can turn in such a manner as indicated by the alternate long and two short dashes line. In the normal light observation mode, the control section 46 drives the motor 72 so as to arrange the DM 71a on the optical path C0. In the special light observation mode, the control section 46 drives the motor 72 so as to arrange the DM 71b on the optical path C0.

Here, the DM 71 has a disc shape, but may have a plate shape.

Furthermore, here, one of the DM 71a and the DM 71b is arranged on the optical path C0 by means of a turning operation of the DM 71 around the shaft 72a of the motor 72, but one of the DM 71a and the DM 71b may be arranged on the optical path C0 by an actuator configured to linearly move DM 71 between two positions.

Therefore, according to the present modification, in the normal light observation mode, a decrease in amount of light reflected by the DM 71a in light from the LED 32d can be prevented.

As described above, the respective embodiments and modification above enable provision of an endoscope apparatus that even if a light source configured to emit illuminating light having a peak wavelength that shifts depending on a value of a drive signal is used, can reduce light having a wavelength unsuitable for desired observation.

Although the respective embodiments and modification described above employ LEDs each providing a peak wavelength that shifts depending on a drive current as light sources, the respective embodiments and modification described above are applicable to cases where an apparatus including solid lasers such as laser diodes, liquid lasers such as dye lasers or gas lasers each providing a peak wavelength that shifts depending on a drive signal is used as a light source.

Still furthermore, although in the above-described first and second embodiments, in the special light observation mode, a plurality of narrow-band light rays are radiated as illuminating light, and the optical filter 51 or the DM 34cA configured not to transmit or reflect light of no more than 595 nm for narrow-band light of 600 nm in the illuminating light is used as band limiting means, light having a predetermined broad band may be used as illuminating light and the color filter 21f in the image pickup section may have a band limiting characteristic of not transmitting light in a wavelength band of no more than 595 nm.

For example, return light from an object enters the image pickup device 21b including the color filter 21f. The color filter 21f includes a blue filter, a green filter and a red filter in, e.g., a Bayer arrangement. For the blue filter, a bimodal filter configured to transmit two narrow-band light rays having respective peak wavelengths of 415 nm and 460 nm is employed, for the green filter, a filter configured to transmit narrow-band light having a peak wavelength of 540 nm is employed and for the red filter, a bimodal filter configured to transmit two narrow-band light rays having respective peak wavelengths of 600 nm and 630 nm is employed. Then, the red filter is provided with a characteristic of transmitting two narrow-band light rays of 600 nm and 630 nm as well as a characteristic of not transmitting light of a wavelength band of no more than 595 nm. Consequently, even if peak wavelength shifting occurs in illuminating light, image quality deterioration can be prevented.

Also, instead of the color filter arranged in front of the image pickup device 21b being provided with a band limitation characteristic of not transmitting light of a wavelength band of no more than 595 nm, a filter section 21g such as indicated by the dotted line in a distal end portion of the light guide 7 in FIG. 1 may be provided and the filter section 21g may be provided with a band limitation characteristic of not transmitting light of a wavelength band of no more than 595 nm.

For example, for the filter section 21g, a pentamodal filter having a characteristic of not transmitting light of no more than 595 nm for narrow-band light having a peak wavelength of 600 nm is employed. Consequently, even if peak wavelength shifting occurs in illuminating light, image quality deterioration can be prevented.

Still furthermore, although in each of the embodiments, a plurality of narrow-band light rays are used as illuminating light, where an image signal obtained from reflected light from an object is subjected to spectrum estimation processing to generate a narrow-band image signal, generation of an image of light of no more than 595 nm may be not done.

For example, in the spectrum estimation processing, an image corresponding to narrow-band light having a peak wavelength of 600 nm is generated so as not to contain a narrow-band image that is based on light in a wavelength band of no more than 595 nm. Consequently, even if peak wavelength shifting occurs in illuminating light, image quality deterioration can be prevented.

As described above, the respective embodiments and modification above enable provision of an endoscope apparatus that even if a light source configured to emit illuminating light having a peak wavelength that shifts depending on a value of a drive signal is used, can reduce light having a wavelength unsuitable for desired observation.

The present invention is not limited to the above-described embodiments, and various changes, alterations and the like are possible without departing from the spirit of the present invention.

What is claimed is:

1. An endoscope apparatus comprising:
a first light emitting section configured to generate, upon supply of a predetermined drive current, light having a peak wavelength at a first wavelength as illuminating light to be radiated to a subject, and to generate, upon supply of a drive current that is different from the predetermined drive current, light with the peak wavelength shifted to a second wavelength that is different from the first wavelength; and
a removal section provided on an optical path for the illuminating light, the optical path extending from the first light emitting section to an image pickup section, the removal section being configured to remove light having a wavelength located farther than the second wavelength in a shifting direction of shifting from the first wavelength toward the second wavelength on a wavelength axis.

2. The endoscope apparatus according to claim 1, wherein in the first light emitting section, the first wavelength is a wavelength within a band of from a wavelength providing a local maximum value to a wavelength providing a local minimum value in a characteristic of absorption by hemoglobin.

3. The endoscope apparatus according to claim 1, wherein the first light emitting section generates, upon supply of the predetermined drive current, narrow-band light having the peak wavelength at a wavelength of no less than 600 nm as the first wavelength, and generates, upon supply of a drive current that is lower than the predetermined drive current, narrow-band light having the peak wavelength at a wavelength of less than 600 nm as the second wavelength.

4. The endoscope apparatus according to claim 3, wherein the removal section removes light having a wavelength of less than 600 nm from the illuminating light.

5. The endoscope apparatus according to claim 3, wherein the removal section removes light having a wavelength of no more than 595 nm from the illuminating light.

6. The endoscope apparatus according to claim 3, wherein the removal section removes light having a wavelength of no more than 591 nm from the illuminating light.

7. The endoscope apparatus according to claim 1, further comprising an observation mode selection section configured to select an observation mode for the subject,
wherein the removal section is inserted/removed onto/from the optical path for the illuminating light, in response to selection of the observation mode via the observation mode selection section.

8. The endoscope apparatus according to claim 1, further comprising:
a second light emitting section configured to generate light having a peak wavelength at a wavelength that is shorter than the wavelength of light generated by the first light emitting section; and
a dichroic mirror arranged on an optical path on which the light generated by the first light emitting section and the light generated by the second light emitting section travel, the dichroic mirror being configured to multiplex the light from the first light emitting section and the light from the second light emitting section,
wherein the removal section is an optical filter provided on an optical path between the first light emitting section and the dichroic mirror.

9. The endoscope apparatus according to claim 1, further comprising a second light emitting section configured to generate light having the peak wavelength at a wavelength that is shorter than a wavelength of light generated by the first light emitting section,
wherein the removal section is a dichroic mirror arranged on an optical path on which the light generated by the first light emitting section and the light generated by the second light emitting section travel, the dichroic mirror being configured to reflect the light from the first light emitting section and transmit the light from the second light emitting section to multiplex the light from the first light emitting section and the light from the second light emitting section.

10. The endoscope apparatus according to claim 1, wherein the removal section is provided on the optical path on which the illuminating light travels from the first light emitting section toward the subject.

11. The endoscope apparatus according to claim 10, wherein:
the illuminating light emitted from the first light emitting section is reflected by a dichroic mirror and radiated toward the subject; and
the removal section is an optical filter arranged between the first light emitting section and the dichroic mirror.

12. The endoscope apparatus according to claim 11, further comprising an observation mode selection section configured to select an observation mode for the subject,
wherein the removal section is inserted/removed onto/from the optical path for the illuminating light, in response to selection of the observation mode in the observation mode selection section.

13. The endoscope apparatus according to claim 7, wherein:
the illuminating light emitted from the first light emitting section is reflected by a dichroic mirror and radiated toward the subject; and
the removal section is the dichroic mirror configured not to reflect light having a wavelength located farther than the second wavelength in the shifting direction.

14. The endoscope apparatus according to claim 13, further comprising the observation mode selection section configured to select the observation mode for the subject,
wherein the dichroic mirror is inserted/removed onto/from the optical path for the illuminating light, in response to selection of the observation mode in the observation mode selection section.
